(19) United States
(12) Patent Application Publication
(10) Pub. No.: US 2020/0248000 A1
(43) Pub. Date: Aug. 6, 2020

(54) A method for manufacturing coated titanium dioxide particles, coated titanium dioxide particles and products comprising thereof

(71) Applicants: Venator P&A Finland Oy, Pori (FI); Venator Germany GmbH, Duisburg (DE)

(72) Inventor: Ralf-Johan Lamminmäki, Ulvila (FI)

(73) Assignees: Venator P&A Finland Oy, Pori (FI); Venator Germany GmbH, Duisburg (DE)

(21) Appl. No.: 16/756,546

(22) PCT Filed: Oct. 15, 2018
(86) PCT No.: PCT/EP2018/078019

§ 371 Date: Apr. 16, 2020

(30) Foreign Application Priority Data
Oct. 17, 2017 (EP) 17196845.6

(51) Int. Cl. C09C 1/36; C01G 23/047; A61K 8/29; A61Q 17/04; C09D 11/037; C09D 7/62

(52) U.S. Cl. CPC C09C 1/3661; C01G 23/047; A61K 8/29; A61Q 17/04; C09D 11/037; C01P 2004/64; C01P 2004/84; C01P 2002/86; C01P 2006/12; C01P 2006/64; C09D 7/62

(57) ABSTRACT

A method for manufacturing pigmentary titanium dioxide particles coated with a SiO₂ coating layer includes steps of (i) forming an aqueous dispersion containing rutile titanium dioxide particles having an average particle size of in a range of 7-1000 nm, (ii) introducing to said dispersion of step (i) a silicon containing compound and a base, (iii) adding to the dispersion obtained from step ii acid, (iv) repeating the steps ii and iii at least once. Finally, the pH of the dispersion is lowered to a value within the range from 1.9 to 9.0 before filtering and washing thus obtained product.

The present disclosure relates to particulate titanium dioxide based material which is coated with silicon oxide. In particular, the coated titanium dioxide is a suitable component for demanding applications, such as a printing ink composition typically used in laminated products.

### BACKGROUND

It is possible to replace a considerable amount of the white titanium dioxide pigment of white ink formulation without losing too much of the optical properties of the white ink. However, there are still problems with this kind of ink especially in solvent based systems wherein the filler is eventually settling in the bottom of the ink container. This settling behaviour is related to the dispersion stability when uncoated fillers are not properly wetted with binder and when they are not stabilized in the system.

There is a clear demand in the printing inks market for a product which has a material cost lower than that of regular rutile titanium dioxide pigment and which is stable in storage conditions. This kind of titanium dioxide based material could be applied in coatings, plastics and paper applications.

Printing inks may be applied as flexible packaging inks, especially lamination inks, and paper and board inks. Laminating inks typically are printed on a clear substrate, which is then laminated by adhesive or molten polymer and “sandwiched” to another material. The gloss level may vary depending on the application; all target markets do not require a high gloss. However, the product particle size distribution should be adjusted to enable rotogravure and flexography printing.

The light scattering performance of titanium dioxide particulate material depends on particle size, particle size distribution and dispersion quality. In an ideal set-up titanium dioxide crystals form a 3-dimensional matrix, where each individual same size round shaped particles are at equal distance apart from each other. This theoretical understanding of light scattering is based on the Mie-theory and depicted by FIG. 1. In reality rutile particles are of varying size and shape and they tend to agglomerate and/or flocculate. However, the scheme of FIG. 1 would represent the ultimate goal for further development.

In the presently commercially available titanium dioxide based material the surface treatment layer is rather thin. To get the best optical properties out of the material, it needs to be wetted and stabilized with a soluble binder and dispersed properly for the particles to stay apart from each other. When white ink is printed the binder solidifies and creates a kind of network of the polymer the function of which is to bind the titanium dioxide particles together and localize them onto the surface. At the same time the binder keeps the particles apart so that good scattering power and increased opacity is achieved.

The portion of the lamination inks is increasing and the formulations are becoming technically more demanding, such as polyurethane formulations. Moreover, competition is getting more challenging as low gloss rutile grades are accepted.

In gravure printing the speeds of the printing machines are increasing. This creates additional requirements for the white inks.

In lamination inks high viscosity polyurethanes and other high molecular weight binders are used. The printing viscosity limits the optical performance of the white ink when both porous pigment and the high molecular weight binder affect the rheology.

When using single polyurethane component coatings together with pigments such as titanium oxide pigments, several problems have been confronted. Typically, the pigments may contain absorbed moisture to some extent causing non-stability in the polyurethane composition. Gelling of the polyurethane may take place, rendering the composition hard and unsuitable for further use. There may be formation of carbon dioxide due to reactions of isocyanate with water building up pressure in the storage vessels.

The prior art fails to provide coated titanium pigment suitable for laminate purposes fulfilling the requirements thereto. Therefore, there is a need for a novel coated titanium dioxide material overcoming the problems discussed above.

### SUMMARY

The object of the present disclosure is to provide particulate titanium dioxide based material which is coated with dense silicon oxide, and which is particularly suitable for use in a lamination ink composition. The present disclosure provides a method for manufacturing titanium dioxide particles coated with a silica coating layer, and coated titanium dioxide particles obtained with said method.

The lamination ink composition comprising the silicon oxide coated particulate titanium dioxide based material is required to deliver high opacity and to have low viscosity.

The present disclosure provides a method for manufacturing a dense silicon dioxide (SiO₂) coating using pH cycling while coating the particulate titanium dioxide based core particles with said SiO₂.

The main embodiments are characterized in the independent claims. Various embodiments are disclosed in the dependent claims. The embodiments recited in the claims and in the description are mutually freely combinable unless otherwise explicitly stated.

One embodiment provides a method for manufacturing non-flocculated discretely distributed titanium dioxide particles coated with a silica coating layer, preferably functioning as a spacer between the individual titanium dioxide particles, the method comprising the steps of

i) forming an aqueous dispersion containing the titanium dioxide particles, wherein the mean particle size, d50, of the titanium dioxide particles is in the range of 7-1000 nm,

ii) introducing to said dispersion a silicon-containing compound under constant mixing, optionally with an addition of a base, to obtain an alkaline dispersion,

iii) adding acid to the alkaline dispersion obtained from step ii to lower the pH to initiate precipitation of silicon oxide from the dispersion onto the titanium dioxide particles, and

iv) repeating the steps ii) and iii) at least once,

to obtain non-flocculated discretely distributed titanium dioxide particles.

The present disclosure further provides silicon dioxide coated titanium dioxide based materials produced by the method disclosed herein.

The silicon dioxide coated titanium dioxide based material is suitable for use in printing laminate ink compositions, in sunscreens and in paint formulations.

The present disclosure further provides compositions, such as a printing laminating ink composition, a sunscreen composition and a paint composition, comprising the silicon dioxide coated titanium dioxide based material.

The method of the present disclosure provides dense coating on the titanium dioxide core. The properties of the coated particles are significantly different compared to TiO₂ particles which are currently commercially available. The enhanced properties include stability of the coated particles in various formulations, BET surface area, oil absorption, undertone and/or tint reducing power of the product. Moreover, the agglomeration or flocculation tendency is decreased. Furthermore, due to the good coverage of the SiO₂ layer on TiO₂, better stability is attained. These improved properties provide advantage in final application where the particles are used. These include better rheology properties for the final ink enabling more freedom to adjust high speed printing properties. Also better strength can be achieved improving lamination properties or enabling lower solvent and/or adhesive demand. Moreover, increased sun protection factor (SPF) can be achieved in sunscreen formulations.

Improved particle coating properties provide an advantage in photostability of TiO₂ crystals giving better durability of coatings in exterior end applications.

The method may be carried out at substantially low temperatures which provide advantages in the process. For example the energy consumption is lower and there is no such need for cooling as would be if higher temperatures were used. There are more options for material selection of the devices, such as the reactor vessel.

### BRIEF DESCRIPTION OF THE FIGURES

FIG. 1 shows an ideal TiO₂ crystal network.

FIG. 2 shows one processing scheme according to the present disclosure.

FIG. 3 shows the NMR spectra of SiO₂ coated titanium oxide particles.

FIG. 4 shows the surface area and oil absorption behaviour of 8% of silica in total deposited on TiO₂ particles.

FIG. 5 shows schematically the structure of a laminate.

FIG. 6 shows the contrast ratios for laminated and unlaminated structures with the SiO₂ coated titanium oxide particles.

FIG. 7 shows that the multiple SiO₂ layer coated TiO₂ samples provided clearly better contrast ratio values than single layer coated samples.

FIG. 8 shows that the multiple SiO₂ layer coated TiO₂ samples provided clearly better contrast ratio values than the single layer coated samples.

FIG. 9 shows the results from a comparison wherein the coated TiO₂ is included into bare films and in laminated films.

FIG. 10 shows how coating the TiO₂ pigment in SiO₂ cycles improves (decreases) the oil absorption and surface area of the particles.

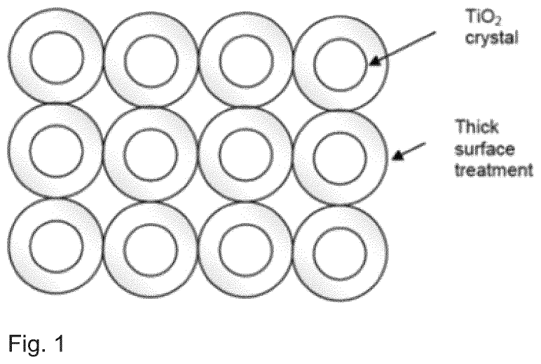

### DETAILED DESCRIPTION

In this specification, percentage values, unless specifically indicated otherwise, are based on weight (w/w; w-%). If any numerical ranges are provided, the ranges include also the upper and lower values.

The silica as used in the present disclosure refers to material which predominantly includes silicon dioxide, SiO₂. However, silica may further contain amounts of hydroxyl groups OH⁻, moisture H₂O and/or hydrogen H⁻ groups.

The non-flocculated discretely distributed particles as used herein refer to single particles which are well separated from each other in a way that these single particles are not in direct contact with each other. The particles are not aggregated i.e. attached to each other or flocculated from the dispersion.

The rutile titanium dioxide as used herein refers to a particular polymorph of the titanium dioxide. Rutile titanium dioxide has a body-centred tetragonal unit cell, with unit cell parameters a=b=4.584 Å, and c=2.953 Å. The titanium cations have a coordination number of 6 meaning that they are surrounded by an octahedron of 6 oxygen atoms. The oxygen anions have a coordination number of 3 resulting in a trigonal planar coordination. Another typical titanium dioxide polymorph is anatase.

The expression “mean particle size” as used herein refers to a volume based mean or median particle size reading obtained from particle slurry measured by a commercially available particle analyser. In the present disclosure Malvern Mastersizer is used. The values for notations d₁₀, d₅₀ or d₉₀ obtained by the particle analyser for particle size distribution are used for describing the mean particle diameter value of the particle size distribution i.e. d₅₀ is the value of the particle diameter at 50% in the cumulative distribution. For example, if d₅₀=5.8 mm, then 50% of the particles in the sample have a mean particle diameter larger than 5.8 mm, and 50% have a mean particle diameter smaller than 5.8 mm. D₅₀ (=d₅₀) is commonly used to represent the particle size of group of particles.

The pigmentary particles as used herein refer to particles that are able to provide hiding power and to impart opacity to a surface. Pigmentary particles of titanium dioxide provide an effective opacifier in powder form, where they are employed as a pigment to provide whiteness and opacity to various range of products. The mean particle size of pigmentary titanium dioxide particles, d₅₀, according to the present disclosure may be within the range of 7-1000 nm, such as in the range of 7-100 nm, in the range of 7-900 or in the range of 100-900 nm.

In one embodiment the so called UV TITAN i.e. transparent titanium dioxide is applied. The transparent UV TITAN refers to titanium dioxide which is transparent and has a mean crystal size is less than 100 nm, and preferably 7 nm or more according to the present disclosure, such as in the range of 7-100 nm. The crystal size refers to the primary particle size without agglomeration.

The first aspect of the present disclosure is a method for manufacturing non-flocculated discretely distributed titanium dioxide particles. The particles are coated with a silica coating layer. The silica coating layer functions as a spacer coating layer between the individual titanium dioxide particles i.e. particulate titanium dioxide based material is provided wherein the titanium dioxide core particles have a dense silicon oxide coating thereon.

The method of the present disclosure comprises the following steps.

(i) Forming an aqueous dispersion containing titanium dioxide particles, wherein the mean particle size, d₅₀, of the titanium dioxide particles is from 7 nm to 1000 nm. The titanium dioxide particles refer to such titanium oxide particles, typically secondary particles, which are obtained directly from a manufacturing process and which have undergone milling for separation or removal of agglomerates or flocculates to form single particles.

(ii) Introducing a silicon-containing compound to the dispersion under constant mixing, optionally with an addition of a base, thus obtaining an alkaline dispersion. The pH as a result of adding the silicon-containing compound may already be alkaline depending on the chemical used, in which case no further addition of a base is necessary. If the dispersion is not alkaline after addition of the silicon-containing compound, a further addition of a base is necessary to render the resulting dispersion alkaline. The pH of the resulting dispersion may be measured using commonly known pH measurement apparatus and techniques.

(iii) Adding acid to the alkaline dispersion obtained from the previous step to initiate precipitation of the silicon-containing compound from the dispersion. By adding acid to the dispersion the pH of the dispersion is lowered to a suitable value enabling silicon compound precipitation from the liquid phase.

(iv) Repeating the steps of adding the silicon-containing compound, with or without the base, and adding the acid at least once. By this pH cycling the precipitation of the silicon compound may be controlled and divided into desired precipitation cycles.

Subsequently, the pH of the dispersion may be lowered with an acid to a value in the range of 1.9-9.0, preferably in the range of 3-8.5, more preferably in the range of 4.5-8, and the obtained product is filtered and washed.

The coating layer containing silicon is deposited onto the surface of the titanium dioxide particles by wet chemical means. By adjusting the conditions of the polar dispersing phase, advantageously aqueous titanium dioxide based dispersion, to a suitable pH range precipitation of the silicon compound is enabled.

The polar dispersing phase is advantageously a polar solvent system, such as water or an aqueous alcohol containing system, whereto the titanium dioxide is readily dispersed.

In an exemplary embodiment the titanium dioxide concentration of the dispersion is in the range of 70-400 g/l. Advantageously, the concentration is in the range of 150-350 g/l, more advantageously in the range of 200-320 g/l, most advantageously in the range of 225-315 g/l, such as in the range of 270-310 g/l. The preferred concentration is high, but the associated viscosity rise causes practical problems for e.g. efficient mixing. The concentrations may be balanced by selecting suitable TiO₂ particle size, used amount thereof and reaction temperature.

In one embodiment the titanium dioxide of the present disclosure exhibits a rutile structure of at least 80% (w/w) or more, preferably 90% (w/w) or more, more preferably 97% (w/w) or more, most preferably 99% (w/w) or more, such as 99.5% (w/w) or more, or even about 100% (w/w), depending on the preparation method thereof.

In one embodiment the so called UV TITAN i.e. transparent titanium dioxide is applied. This titanium dioxide exhibits at least 80% (w/w) rutile structure.

In one embodiment, in the first method step an aqueous dispersion is formed containing at least 97% (w/w) of rutile form titanium dioxide particles having a mean particle size in the range of 100-1000 nm, such as in the range of 100-900 nm. The particle shape is advantageously spherical. Occasionally, the particles may be acicular in shape and in such case the largest dimension of the particles may be in the range of 100-800 nm. The ratio of the largest dimension to the shortest dimension may be from 2:1 to 3:2. Advantageously, the particles further have a narrow size distribution; at least 80 percent by weight have a size within the range of mean particle size of in the range of 200-300 nm.

In an exemplary embodiment the mean particle size, d₅₀, of the rutile titanium dioxide particles is at least 150 nm, advantageously at least 175 nm, such as at least 200 nm.

In another exemplary embodiment the mean particle size, d₅₀, of the rutile titanium dioxide particles is less than 450 nm, advantageously less than 400 nm, such as less than 300 nm. In some embodiments the mean particle size, d₅₀, of the rutile titanium dioxide particles is in the range of 150-450 nm, such as 150-400 nm, 175-400 nm, 175-450 nm, 200-400 nm, or 200-450 nm.

A uniform coating is provided by mixing the dispersion during pH cycling. Especially, a dense silica coating is aimed at. The term dense as used herein refers to a coating which shows clearly modified characteristics or properties in comparison with particles including regular surface treatments. For example, the quality of the coating may be evaluated by changes in the oil absorption properties of the surface. In addition, the change in the coating layer can also be seen in other properties of the product, such as in filtration and washing times during the production process, and in the specific surface area (BET) values, total pore volume and average pore radius of the coated pigment. Indirectly, the density of the surface influences the properties of a laminate and a printing ink composition using such coated material.

The particulate titanium dioxide based material of the present disclosure may be formed by any suitable process. Advantageously, it is manufactured by a sulphate process as depicted by EP0444798B1 or EP0406194B1. Most preferably, the microcrystalline or UV-TITAN i.e. TiO₂ particles with a particle size 100 nm or less are manufactured according to the example 1 of EP0444798B1, and pigmentary TiO₂ with a particle size more than 100 nm according to the example 1 of EP0406194B1.

Usually prior to coating the particulate titanium dioxide based material is preferably milled to an appropriate particle size falling within the desired range employing grinding medium such as sand which can be separated easily and effectively from the milled product. Milling may be carried out in the presence of a dispersing agent such as sodium silicate or another dispersant, for example an organic dispersant, such as monoisopropanolamine(1-amino-2-propanol). Wet milling may be performed by regular milling means known in the art, such as bead milling.

In an exemplary embodiment the temperature of the titanium dioxide containing dispersion is maintained at a value in the range of 40-100° C. Advantageously, the temperature of the dispersion in the range of 50-90° C. to enable use of varying container materials, more advantageously in the range of 60-85° C., or 60-80° C., for efficient energy consumption, most advantageously in the range of 63-80° C. or 63-75° C., such as about 65° C. A lower temperature is preferred due to faster cooling time before possible subsequent washing. The dispersion may be externally heated to maintain the optimal reaction temperature using regular heating means. Moreover, the dispersion is mixed using regular means for mixing to maintain homogeneity and to provide a uniform coating.

In the second step the silicon-containing compound, and optionally a base, is introduced to said dispersion of the titanium dioxide particles, such as rutile titanium dioxide particles.

In an exemplary embodiment of the present disclosure the silicon-containing compound to be used as coating agent is any suitable water soluble silicate. Advantageously, an alkali metal silicate is employed. Particularly useful are sodium and potassium silicates, and most advantageously the solution of the silicate is freshly prepared prior to application.

In a further exemplary embodiment the silicon-containing compound to be used as a precursor for the coating is selected from the group consisting of water glass, silica sol, SiO₂, and an organic silicon compound. The organic silicon compound preferably comprises ortosilicate or tetraethylortosilicate. The silica sol refers to colloidal silica having a chemical molecular formula of mSiO₂.nH₂O. It is odourless, tasteless and nontoxic. Most advantageously, water glass is applied. It is commercially readily available and efficient chemical, and its aqueous solution is stable enough for the present application.

In another exemplary embodiment the base to be added into the dispersion before, after or during the addition of the silicon containing compound is used for increasing the pH of the dispersion to a value wherein the silicon compound remains in dissolved form. Advantageously, the base is selected from the group consisting of NaOH, KOH, Na₂CO₃ or ammonia. In particular, it is advantageous to add NaOH, Na₂CO₃ or ammonia, most preferably NaOH. These bases do not introduce any additional ionic species into the dispersion. The base is preferably added as a concentrated aqueous solution.

In an exemplary embodiment the pH of the dispersion after addition of the silicon-containing compound, with or without the addition of base, is in the range of 9.3-12. Advantageously, the pH is in the range of 9.5-11 to ensure proper dissolution of the silicon in the aqueous phase.

In an exemplary embodiment the silicon-containing compound is added in an amount in the range of 50-100 g/l, preferably in the range of 55-90 g/l, more preferably in the range of 60-80 g/l, calculated as SiO₂. This addition is in relation to the addition of the titanium dioxide. Advantageously, if n is the number of SiO₂ cycles and y is the total amount of SiO₂, the amount of silicon contained in the layer, x, is x=y/n.

In an exemplary embodiment the amount of silicon in one layer is 3% (w/w) wherein the amount of titanium dioxide is 94% (w/w) provided that the number of layers is 2.

Subsequently, in the third step of the method according to the present disclosure acid is added to the dispersion. The purpose of the acid addition is to lower the pH and initiate and maintain the precipitation of the silicon oxide onto the titanium dioxide particles. The precipitation of silica results from the addition of a mineral acid to an alkaline solution of the soluble silicate and titania to hydrolyse the silicate in solution to dense silica.

In an exemplary embodiment the pH after addition of the acid is in the range of 4-9.3, such as in the range of 4-9 or 4-8.5, advantageously in the range of 4.3-8.5 or 4.3-8, more advantageously in the range of 4.5-7.8, most advantageously in the range of 5-7.5, such as about 7.3. The upper pH limit restricts the precipitation. At the acidic end the viscosity increases decreasing the capacity. If water is added the concentration is decreased which is typically an undesired feature.

In an exemplary embodiment the acid is selected from inorganic mineral acids or organic acids. Advantageously, the acid comprises sulfuric acid, nitric acid, hydrogen chloride, formic acid, acetic acid or oxalic acid. In particular, the preferred acid is sulphuric acid, such as concentrated sulphuric acid, wherein no additional ionic species need to be introduced into the process.

In the method of the present disclosure the pH of the dispersion is subsequently increased again into the silicon dioxide dissolution range i.e. the dissolution step is repeated by adding further base into the dispersion, preferably together with additional silicon-containing compound. The pH increase further enables dissolution of the already formed silicon dioxide coating layer, more particularly the less dense outer part of the formed coating. The pH cycling is also repeated by further addition of a portion of the acid, thus lowering the pH of the dispersion back to the silicon dioxide precipitation range. The dissolution and precipitation steps ii and iii are repeated at least once, preferably at least twice.

In one embodiment the steps ii and iii are repeated at least two times. In one embodiment the steps ii and iii are repeated at least three times. In one embodiment the steps ii and iii are repeated at least four times. In one embodiment the steps ii and iii are repeated at least five times. Especially, when transparent UV TITAN is coated more coating cycles are advantageous. In one embodiment the steps ii and iii are repeated at least six times, especially when heavily coated transparent titanium is needed.

In an exemplary embodiment there is a delay or residence time in the dissolution and precipitation steps. This time is required for each reaction to take place is advantageously least one minute, more advantageously at least two minutes, most advantageously at least three minutes to ensure efficient mixing and controlled dissolution or precipitation reactions, and to provide a sharp change in the reaction conditions of the dispersion pH. In some examples the delay or residence time is in the range of 1-30 minutes, 2-30 minutes, 3-30 minutes, 1-10 minutes, 2-10 minutes, 3-10 minutes, 1-5 minutes, 2-5 minutes or 3-5 minutes.

It is anticipated without being bound by any theory that the formation of the Si—O bonds is enhanced by the cycling procedure of the present disclosure. Thus, a very dense SiO₂ coating or a coating comprised of multiple coating layers is produced. By direct single precipitation cycle a fluffy Si—O network is formed with an oxygen deficiency. By cycling the pH enabling multiple dissolution and precipitation cycles a denser i.e. glassy Si—O network is achieved. In this network the amount of oxygen corresponds to multiple, such as tetravalent coordination of Si—O.

The dense SiO₂ coating layer of the present disclosure enables a smaller product particle size. As the total diameter of the particulate product is decreased the dispersing ability is increased and the optical efficiency is increased. Moreover, the wettability of the particle is better and its concentration may be increased.

In the present disclosure the coating sequence is pH controlled comprising interruptions in between the SiO₂ coating formation i.e. the coating is performed stepwise. This multistep coating comprising precipitation and dissolution cycling of silicon dioxide results in formation of a dense SiO₂ multilayer on top of the titanium dioxide core material. The resulting coating of dense silica is substantially non-porous, amorphous and continuous around the titanium dioxide particles.

The dense amorphous silica when present in the form of a coating on the particles forms a barrier between the titanium dioxide particles and the medium in which the titanium dioxide particles are dispersed and reduces, for example, migration of reactive species from the particles to the medium or vice versa. Dense amorphous silica is formed under controlled precipitation conditions which are described above. The particles of the present disclosure may be coated with widely differing amounts of the dense amorphous silica.

In one embodiment the amount of SiO₂ is in the range of 2-25% (w/w), such as in the range of 4-10% (w/w), of the coated product.

After the deposition of the multiple silicon containing layers on top of the titanium dioxide based core material is ready, the product preparation is finalized by lowering the pH of the dispersion to a value in the range of 4.5-8, preferably in the range of 4.5-5.5, before filtering and washing the product thus obtained.

A slightly acidic product pH is preferred for the end product to remove the traces of sodium from the surface. The subsequent washing removes the impurities and the product may be further dried, grinded, and optionally coated by regular means with for example with an organic layer.

In an exemplary embodiment the organic layer comprises deposition of large-molecule fatty acid salts, organic silicon compound such as silicone oil, alkyl silane, olefinic acid, polyol, dimethyl polysiloxane, alcohol, polyalcohol, organophosphonic acid, such as dimethicone and/or dibenzoyl methane derivative onto the silicon dioxide coated titanium dioxide particle.

The manufacturing process of the present disclosure differs from the prior art silicon dioxide coating processes in that multiple coating layers of the single or same SiO₂ material are produced using pH cycling.

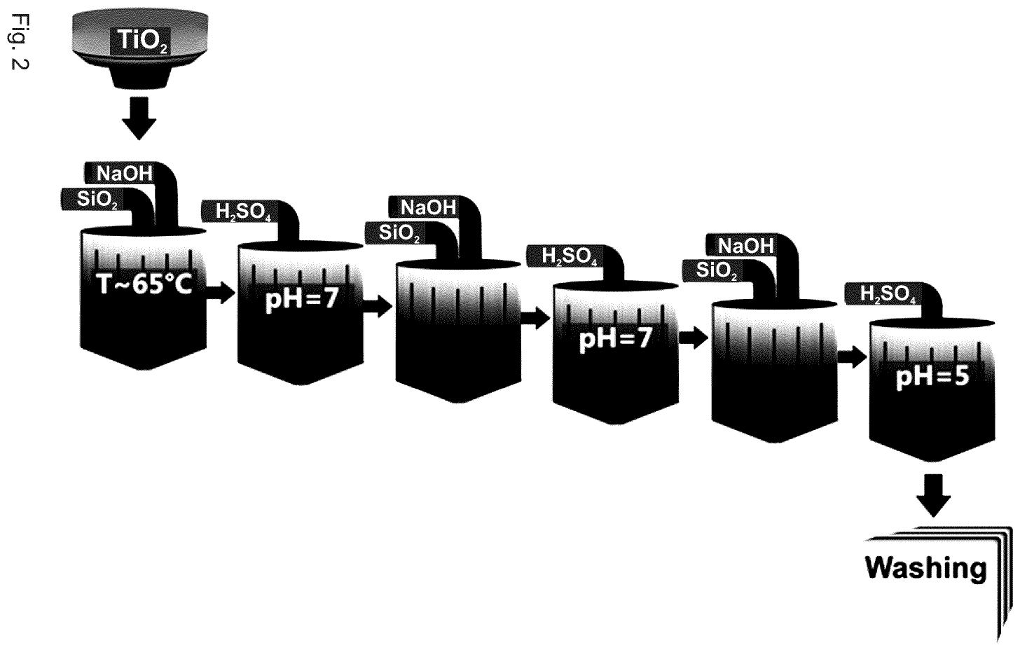

In an exemplary embodiment a preparation process as depicted by FIG. 2 for a 3-layered silicon dioxide coating on the titanium dioxide particles is applied. The base or core titanium dioxide from the manufacturing process thereof is directed to a feed vessel. In the first pH adjustment cycle, the silicon containing compound solution, such as water glass, together with a base, such as NaOH, are introduced into the titanium dioxide dispersion vessel 1. The content of the vessel is mixed for obtaining a homogeneous solution, and the resulting dispersion slurry is further directed to the vessel 2. Acid, such as sulfuric acid is introduced into vessel 2, lowering the pH of the dispersion slurry into a range suitable for precipitation of silicon compound. The content of the vessel is further mixed for a suitable time to ensure homogeneity, and the dispersion slurry is subsequently directed to vessel 3 for a further addition of the silicon containing compound and base. The pH is increased into a range wherein the silicone compound is dissolved. The resulting slurry in subjected to further acidification in vessels 4 and 6, and for a further addition of the silicon containing compound and base in vessels 3 and 5. Finally, the pH of the resulting product slurry is lowered to a targeted product value, and the finished product of titanium dioxide coated with a dense silicon dioxide layer is obtained, and preferably filtered, washed and dried.

As the second aspect, the present disclosure provides a coated titanium dioxide product suitable, in particular, for printing ink applications. This product is manufactured by the above described method.

In one embodiment the product comprises at least 97% of rutile form titanium dioxide core particles coated with a SiO₂ spacer coating layer, having a mean particle size, d₅₀, of from 200 to 300 nm, wherein said product has ²⁹Si chemical shift peaking at (−105)-(−115) ppm in solid state NMR (nuclear magnetic resonance) spectrum indicating fully symmetric Si—O—Si bonding. The titanium oxide based product coated with a dense SiO₂ coating layer is especially suitable for use in demanding applications such as in printing ink application. In particular, the targeted application of this dense silica coated product is in lamination inks and/or reverse printing inks. In both of these applications heavily coated TiO₂ volume is presently used.

In one embodiment the amount of the SiO₂ spacer coating layer in the above product is in the range of 2-4% (w/w) of the coated titanium dioxide product.

The pigmentary product in the range of 200-300 nm obtained by the presently disclosed method is novel as it shows characteristics and properties that have not been found in the prior art products. The formation of a dense silicon dioxide coating is supported by analytical measurements in comparison with literature data and properties measured for products commercially available.

In another embodiment the amount of the SiO₂ spacer coating layer in the above product is in the range of 2-14% (w/w) of the coated titanium dioxide product. This type of coated titanium dioxide is especially well suited for paint formulations.

In an exemplary embodiment a coated titanium dioxide product is provided wherein the titanium dioxide product has a BET surface area which is less than 20 m²/g, such as less than 15 m²/g, preferably less than 12 m²/g. BET values disclosed here are defined based on measurements made using Micromeritics Tristar II 3020 specific surface meter, serial no. 1319 (commissioning date Nov. 13, 2014, from Oy G. W. Berg & Co Abby)

In an exemplary embodiment a coated titanium dioxide product is provided wherein the titanium dioxide product has oil absorption less than 30%, preferably less than 28%. The oil adsorption values disclosed herein are measured according to ASTM D281-95(2007) Standard Test Method for Oil Absorption of Pigments by Spatula Rub-out, using crude linseed oil having an acid value 3±1 (ASTM).

In an exemplary embodiment a coated titanium dioxide product is provided wherein the titanium dioxide product has a tint reducing power L* (gray paste) more than 64.

In an exemplary embodiment a coated titanium dioxide product is provided wherein the titanium dioxide product has an undertone b* less than −6.

Tint reducing power (L*) refers to the ability of pigment to lighten the colour of a black or coloured paint or paste. Undertone (b*) refers to the tint tone of the paint or paste containing titanium dioxide pigment. Determination of the values herein includes a measurement of intensity of reflected light from a sample film on a plastic chart. Tinting strength and undertone are calculated from X, Y, Z values and given as L*, a*, b* values according to CIE LAB system using Hunterlab UltraScan XE colour meter.

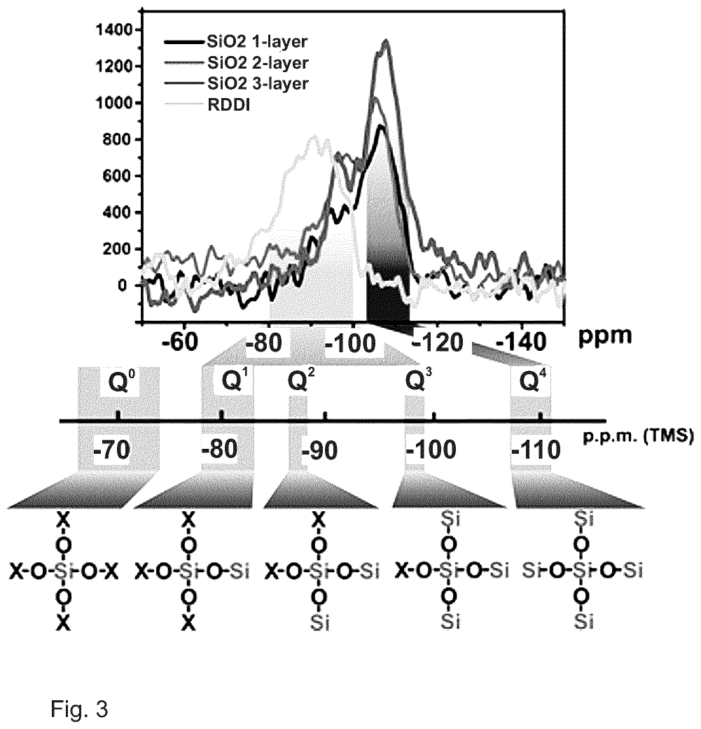

While studying the solid state nuclear magnetic resonance (NMR) spectra obtained from the dense silicon dioxide coated titanium dioxide particles it was realized that the multi-layered or recycled SiO₂ coating structure resulted in enhanced peak intensity together with a peak position change towards negative ppm values. These shifts may be allocated to different structural units of silicate anions in solid silica, for example emerging in the range from −80 to −110 ppm (TMS) as depicted by FIG. 3. The commercial sample (RDDI) exhibits a peak in the range of values from −80 to −100, whereas the SiO₂ coating has the main peak at about −110, or at about −115. As the number of the SiO₂ layers increase the position is shifted towards more negative values. The peak positions are allocated to structural changes of increasing order of Si—O bonds from left to right

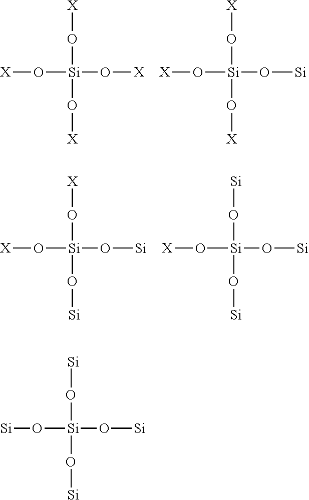

wherein the 5-Si-coordination may be attributed to the multi-layered dense SiO₂ coating structure. The ²⁹Si chemical peak has shifted from the range of values from −80 to −100 ppm towards values less than −100 ppm, such as to about −105 ppm, to about −110 ppm, or to about −115 ppm, or less, such as to about −120 ppm in solid state NMR.

The dense SiO₂ multilayer provides enhanced properties for the TiO₂ pigment particle resulting in enhanced performance of the printing ink comprising these pigments, especially in laminated paper use.

In an exemplary embodiment silica surface treated rutile titanium dioxide particles were prepared according to the method of the present disclosure. This pigment is in accordance with the following generally known classification specifications: ISO 591, DIN 55912, CAS no. (TiO₂) 13463-67-7, ASTM D476 III, EINECS no. (TiO₂) 2366755, Colour index 778891, Components listed in TSCA, EINECS, Pigment White 6.

The product has the following typical properties:

Refractive index 2.7

Relative tint reducing power 1800

Oil absorption (g/100 g pigment) approximately 30

TiO₂ content (%) at least 89

Surface treatment SiO₂, organic coating layer

pH approximately 8.0

Moisture when packed (%) maximum 0.9

Crystal size (mean) (nm) approximately 240

Specific gravity (g/cm³) 3.9

Bulk density (kg/m³) approximately 500

Bulk density (tamped) (kg/m³) approximately 600.

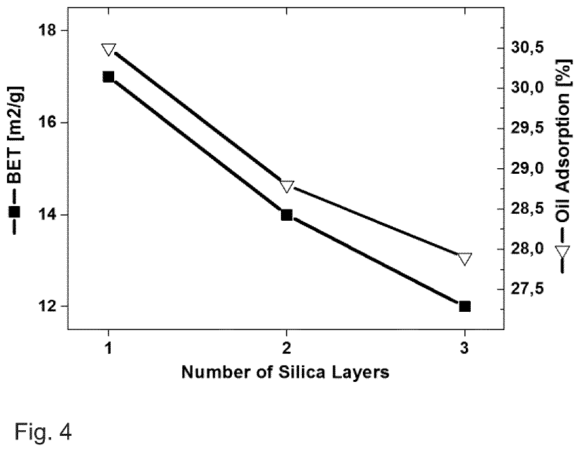

In an exemplary embodiment, the oil absorption of the silica coated titania decreases from a value of 30% or more to less than 28% when the number of the silica layers is increased from 1 to 3. At the same time the surface area, BET is decreased from about 17 m²/g to about 4 when the number of the silica layers is increased from 1 to 3 as is shown in FIG. 4.

As the third aspect, the present disclosure provides use of the products obtained by the method of the present disclosure.

As the fourth aspect, the present disclosure provides products comprising the coated titanium dioxide obtained by the method of the present disclosure.

The product obtained by the presently disclosed method is a heavily coated pigmentary TiO₂ particle which has a low pore volume. The use of this product in printing ink composition improves rheology and leads to higher opacity in printing viscosity.

In yet further embodiment the product comprises at least 80% (w/w) of rutile form titanium dioxide core particles coated with a SiO₂ spacer coating layer, having a mean particle size, d₅₀, less than 100 nm, preferably from 7 to 100 nm, wherein said product has ²Si chemical shift peaking at (−105)-(−115) ppm in solid state NMR (nuclear magnetic resonance) spectrum indicating fully symmetric Si—O—Si bonding. The transparent titanium oxide based product coated with a dense SiO₂ coating layer is especially suitable for use in sunscreen applications.

In one embodiment the product suitable for sunscreen application has the amount of the SiO₂ spacer coating layer in the range of 4-25% by weight of the coated titanium dioxide product.

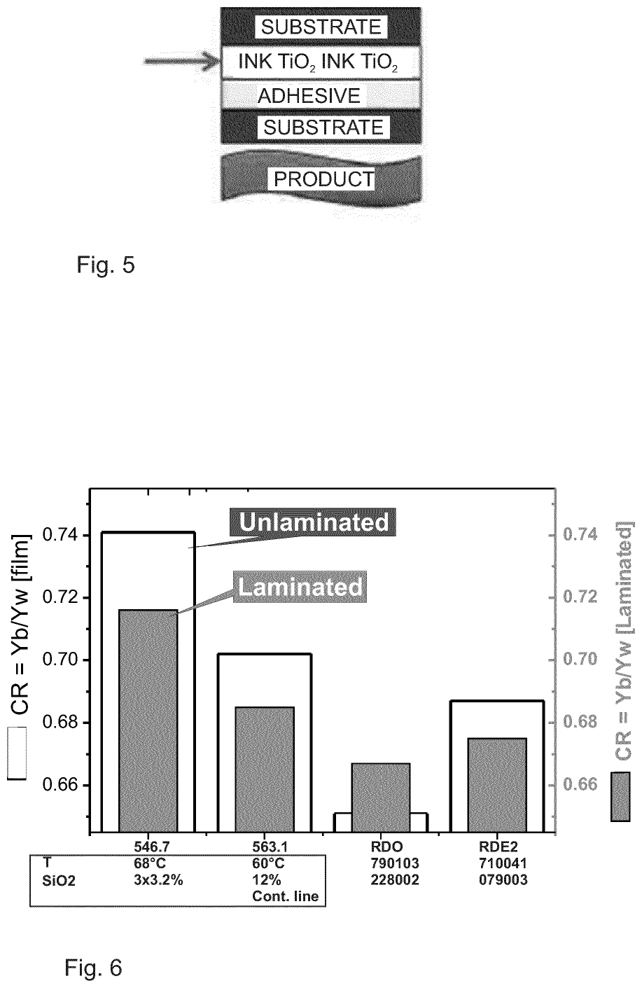

The product of the present disclosure is especially well suited for use in printing ink applications, especially for reverse and lamination printing. As it has essentially no gloss it may be used in matt surfaces. The narrow particle size distribution renders it suitable for high quality flexographic and gravure printing. FIG. 5 depicts the structure of a laminated application including the printing ink composition comprising the dense silicon dioxide coated titanium dioxide material.

One embodiment provides a printing ink composition comprising the coated titanium dioxide product. The printing ink composition may be a lamination ink composition (also called as laminating ink), or a reverse ink composition. The printing ink composition usually contains one or more solvent(s), binder(s), filler(s), other pigment(s), rheological additive(s) and/or the like ingredients commonly used in the art.

For example, in gravure printing the speeds are increasing. This creates additional requirements for the white ink formulations.

In lamination ink compositions high viscosity polyurethanes and/or other high molecular weight binders are generally used. The printing viscosity limits the optical performance of the white ink when both porous pigment and the high molecular weight binder effects the rheology. Using the low pore volume high opacity product of the present disclosure these challenges can be solved.

In lamination inks no gloss is needed. The gloss comes from the plastic substrate on top of the packaging material. In lamination ink it is possible to use more heavily coated particles which destroys the gloss but improves the opacity. The limit of the rough particles (d₉₀) is about 2 μm to enable good runnability on the printing machine.

With minimizing the pore volume it is possible to improve the rheology. Also the low pore volume of the pigment improves the adhesion and bond strength inside the laminate structure.

The product of the present disclosure is able to deliver high opacity and low viscosity in polyurethane system.

In an exemplary embodiment a lamination printing ink composition is obtained comprising titanium dioxide particles coated with a dense silica multilayer with more than 5% SiO₂ and surface area below 12 m²/g and oil adsorption less than 30%.

The dense silica multilayer coated titania when incorporated into lamination printing ink is able to provide high opacity both before and after lamination in the end application. The contrast ratio is increased at least 50% compared single silica layer coated titania as depicted by FIG. 6.

In these applications heavily coated TiO₂ is presently used. The product of the present disclosure improves the rheology and leads to higher opacity in printing viscosity due to the specifically low pore volume provided by the dense SiO₂ coating on top of the titanium dioxide based core material.

Heavily silica coated TiO₂ particles decreases the IEP (isoelectric point) of the pigment product. If needed, the IEP can be adjusted higher by introducing alumina layer on top of the silica coated particles by means of conventional precipitation methods used commonly by the pigment industry. Therefore in one embodiment the non-flocculated discretely distributed titanium dioxide particles which are coated with a silica coating layer comprise an alumina layer on top of the particles.

One embodiment provides a sunscreen composition comprising the coated titanium dioxide product. The coated titanium dioxide product acts as an inorganic particulate active ingredient, which is combined with a carrier, such as a lotion, spray, gel or other topical product

In one embodiment the use a coated transparent titanium dioxide product suitable for sunscreen applications is provided wherein the product is manufactured by the method of the present disclosure comprising at least 80% of rutile form transparent titanium dioxide core particles coated with a SiO₂ spacer coating layer, having a mean particle size less than 100 nm, wherein said product has ²⁹Si chemical shift peaking at (−105)-(−115) ppm in solid state NMR (nuclear magnetic resonance) spectrum indicating fully symmetric Si—O—Si bonding. Preferably, the amount of the SiO₂ spacer coating layer on the transparent titanium dioxide product is in the range of 4-10% (w/w) of the coated titanium dioxide product.

One embodiment provides a paint or a coating composition comprising the coated titanium dioxide product. The coating composition usually contains one or more solvent(s), binder(s), filler(s), other pigment(s), rheological additive(s) and/or the like ingredients commonly used in the art.

One embodiment provides a plastic material or a plastic product comprising the coated titanium dioxide product. The titanium dioxide product may be incorporated into plastic, such as into plastic fibres. The titanium dioxide product may change the properties of the plastic and may be used to obtain a pigmented plastic.

The present disclosure is further illustrated by the following nonbinding examples.

### EXAMPLES

### NTU (Turbidity)

The turbidity is expressed by nefelometric turbidity unit NTU. It was measured by turbidimeter HACH 2100 in a 30 ml cuvette.

### SPF

SPF denotes sun protection factor and was measured from a homogenized emulsion using Labsphere's UV-2000S Ultraviolet Transmittance SPF analyser.

### C Vitamin (Colour Change)

The chemical stability of microcrystalline TiO₂ is assessed utilizing vitamin C colour change measurement. Vitamin C changes colour in the presence of unstable TiO₂. The measurement is typically performed either in oil or in water based medium detecting the colour change by a colour meter, such as Minolta Chroma Meter CR-410.

### Parsol (Colour Change)

Stability of TiO₂ is further studied using a colour change measurement of Parsol 1789 (avobenzone) detected by Minolta Chroma Meter CR-410.

### PG (Photo Graying)

The photocatalytic activity of TiO₂ in a cosmetic emulsion is determined by the percentage of the ΔE value according to the CIE L*a*b system of the presumably photocatalytic TiO₂ sample in regard to ΔE value of the corresponding non-photocatalytic TiO₂ sample. Minolta Chroma Meter CR-410 was used for determination of the CIE coordinates with ATLAS SUNTEST CPS+ as irradiation source.

Bulk Density TP₀, TP₁₀₀ and TP₆₀₀

The bulk density is determined by inserting the material to be evaluated into a column. If the material is inserted loosely the value TP₀ indicates the bulk density, wherein a low value is a measure of high density and a high value depicts low density. TP100 is measured by tapping the column for 100 times, and TP600 is measured by tapping the column for 600 times.

### NMR

The solid state nuclear magnetic resonance (NMR) spectra were recorded using Brooker AV400 (400 MHz) equipment having magic angle spinning 12 kHz. Detection elements were ²⁷Al (5/2) and ³¹P(1/2) and ²⁹Si(1/2), and measurement parameters 1 μs pulse, relaxation delay 0.1 s/10 s. Solid state samples were measured using alumina and silica without TiO₂ as reference.

### Example 1

Titanium dioxide was prepared using a sulphate process according to the method disclosed in EP0406194B1, example 1. This product was subsequently wet milled into a slurry having TiO₂ concentration of about 295 g/l. The particle size distribution of the twice wet milled base slurry was d₁₀=0.179; d₅₀=0.347; d₉₀=0.656 μm.

A 3-layered silicon dioxide coating was manufactured onto the titanium dioxide core particles.

First, the core titanium dioxide particles were directed to the first feed vessel. The temperature of the reaction vessels was maintained at 80° C. The pH of the slurry was 9.1

Subsequently, silica was introduced into the vessel in form of water glass solution (64 g/l SiO₂), and the pH of the vessel was regulated using 25 w-% H₂SO₄ and 30 w-% NaOH in the following way:

1) Adding the first 3.2 w-% SiO₂—pH was measured to be 9.8.

2) pH was adjusted with H₂SO₄ to 7.3 and mixed for 18 min.

3) Adding the second 3.2 w-% SiO₂—pH was measured to be 9.6.

4) pH was adjusted with H₂SO₄ first to 9.5 and mixed for 1 min.

5) pH was adjusted with H₂SO₄ first to 9.0 and mixed for 12 min.

6) pH was adjusted with H₂SO₄ first to 7.3 and mixed for 6 min.

7) Adding the third 3.2 w-% SiO₂—pH was measured to be 9.5.

8) The slurry was mixed 10 min—pH was measured to be 9.5.

9) pH was adjusted with H₂SO₄ first to 9.0 and mixed for 10 min.

10) pH was adjusted with H₂SO₄ first to 7.3 and mixed for 5 min.

11) pH was adjusted with NaOH to 7.6.

After the three-layered coating with SiO₂ was deposited, the particles were subjected to addition of 0.5 w-% P₂O₅ (97 g/l) in the form of Calgon (Merck). The resulting slurry was mixed, cooled down to 60° C. and filtrated. The formed cake was washed and dried at 105° C. At this stage the photostability and BET measurements were performed. Subsequently, the surfaces of the formed particles were coated by introducing 0.1 w-% TMP.

The results of the samples in terms of oil absorption, SiO₂ amount, b*, BET and bulk density TP₀, TP₁₀₀ and TP₆₀₀ are presented in Table 1.

TABLE 1

| | |
|---|---|
| oil absorption | 23.1 |
| SiO₂ amount | 7.27 |
| b* | −6.67 |
| BET | 10 |
| TP₀ | 615 |
| TP₁₀₀ | 739 |
| TP₆₀₀ | 761 |

### Example 2

Titanium dioxide was prepared using a sulphate process according to the method disclosed in EP0444798B1, example 1. This product was subsequently wet milled into slurry having TiO₂ concentration of about 222 g/l. The particle size distribution of the twice wet milled base slurry was d₁₀=0.015; d₅₀=0.100; d₉₀=0.025 μm.

A 3-layered silicon dioxide coating was manufactured onto the titanium dioxide core particles.

First, the core titanium dioxide particles were directed to the first feed vessel. The temperature of the reaction vessels was maintained at 80° C. The pH of the slurry was 9.9

Subsequently, silica was introduced into the vessel in form of water glass solution (64 g/l SiO₂), and the pH of the vessel was regulated using 25 w-% H₂SO₄ and 30 w-% NaOH in the following way:

1) Adding the first 7.0 w-% SiO₂—pH was measured to be 9.9.

2) pH was adjusted with H₂SO₄ to 7.3 and mixed for 15 min.

3) Adding the second 7.0 w-% SiO₂—pH was measured to be 9.8.

4) pH was adjusted with H₂SO₄ first to 9.5 and mixed for 3 min.

5) pH was adjusted with H₂SO₄ first to 9.0 and mixed for 10 min.

6) pH was adjusted with H₂SO₄ first to 7.3 and mixed for 7 min.

7) Adding the third 7.0 w-% SiO₂—pH was measured to be 9.7.

8) The slurry was mixed 10 min—pH was measured to be 9.7.

9) pH was adjusted with H₂SO₄ to 9.5 and mixed for 4 min.

10) pH was adjusted with H₂SO₄ to 9.0 and mixed for 11 min.

11) pH was adjusted with H₂SO₄ to 7.3 and mixed for 6 min.

12) pH was adjusted with NaOH to 7.6.

After the three-layered coating with SiO₂ was deposited, the particles were subjected to addition of 0.5 w-% P₂O₅ (97 g/l) in the form of Calgon (Merck). The resulting slurry was mixed, cooled down to 60° C. and filtrated. The formed cake was washed and dried at 105° C. At this stage the photostability and BET measurements were performed. Subsequently, the surfaces of the formed particles were coated by introducing 6.0 w-% PDMS (poly(dimethylsiloxane)) emulsion.

### Comparative Example 1

Titanium dioxide was prepared using a sulphate process according to the method disclosed in EP0444798B1, example 1. This product was subsequently wet milled into slurry having TiO₂ concentration of about 222 g/l. The particle size distribution of the twice wet milled base slurry was d₁₀=0.015; d₅₀=0.020; d₅₀=0.025 μm.

A 1-layer silicon dioxide coating was manufactured onto the titanium dioxide core particles.

First, the core titanium dioxide particles were directed to the first feed vessel. The temperature of the reaction vessels was maintained at 80° C. The pH of the slurry was 9.9

Subsequently, silica was introduced into the vessel in form of water glass solution (64 g/l SiO₂), and the pH of the vessel was regulated using 25 w-% H₂SO₄ and 30 w-% NaOH in the following way:

1) pH was adjusted with NaOH to 10.4.

2) The slurry was mixed 30 min—pH was measured to be 10.5.

3) Adding 21.0 w-% SiO₂—pH was measured to be 9.9.

4) The slurry was mixed 20 min—pH was measured to be 9.8.

5) pH was adjusted with H₂SO₄ slowly to 9.5 and mixed for 30 min.

6) pH was adjusted with H₂SO₄ to 7.3 and mixed for 30 min.

After the coating with SiO₂ was deposited, the particles were subjected to addition of 0.5 w-% P₂O₅ (97 g/l) in the form of Calgon (Merck). The resulting slurry was mixed, cooled down to 60° C. and filtrated. The formed cake was washed and dried at 105° C. At this stage the photostability and BET measurements were performed. Subsequently, the surfaces of the formed particles were coated by introducing 6.0 w-% PDMS emulsion.

### Example 3

The results of the samples from example 6 and comparative example 1 in terms of NTU, SPF, C vitamin, Parsol, PG, oil absorption, BET and bulk density TP₀, TP₁₀₀ and TP₆₀₀ are presented in table 2.

TABLE 2

| | Example 3 | Comparative example 1 |
|---|---|---|
| NTU | 10.1 | 16.9 |
| SPF | 27.5 | 25.4 |
| C vitamin | 13.4 | 13.5 |
| Parsol | 10.4 | 14.2 |
| PG | 0.5 | 2.8 |
| oil absorption | 65.7 | 74.0 |
| BET | 42.8 | 43.7 |
| TP₀ | 87 | 113 |
| TP₁₀₀ | 95 | 118 |
| TP₆₀₀ | 104 | 136 |
| P₂O₅ % | 0.004 | 0.004 |
| Rutile % | 99.9 | 99.9 |
| SiO₂ % | 20.374 | 20.262 |

Particularly good results were obtained for the photograying wherein the sample according to example 6 is clearly very passive compared to the single SiO₂ barrier layer sample. The colour change is better in example 6 compared to the single, especially in view of Parsol-test. The sun protection factor is higher providing better protection with the same amount of material. The product of sample 6 is also clearly more hydrophobic compared to the single SiO₂ barrier layer sample as the NTU turbidity value is considerably lower.

### Example 4

Titanium dioxide was prepared using a sulphate process according to the method disclosed in EP0406194B1, example 1. This product was subsequently wet milled into a slurry having TiO₂ concentration of about 300 g/l. The particle size distribution of the twice wet milled base slurry was d₁₀=0.207; d₅₀=0.376; d₉₀=0.703 μm.

A 4-layered silicon dioxide coating was manufactured onto the titanium dioxide core particles.

First, the core titanium dioxide particles were directed to the first feed vessel. The temperature of the reaction vessels was maintained at 65° C. The pH of the slurry was 9.4.

Subsequently, silica was introduced into the vessel in form of water glass solution (63 g/l SiO₂), and the pH of the vessel was regulated using 25 w-% H₂SO₄ and NaOH in the following way:

1) pH was adjusted with H₂SO₄ to 8.0 and the slurry was mixed for 5 minutes.

2) Adding the first 2.0 w-% SiO₂—pH was measured to be 9.7.

3) pH was adjusted with H₂SO₄ to 8.0 and the slurry was mixed for 10 minutes.

4) Adding the second 2.0 w-% SiO₂—pH was measured to be 9.6.

5) pH was adjusted with H₂SO₄ to 8.0 and the slurry was mixed for 10 minutes.

6) Adding the third 2.0 w-% SiO₂—pH was measured to be 9.5.

7) pH was adjusted with H₂SO₄ to 8.0 and the slurry was mixed for 10 minutes.

8) Adding the fourth 2.0 w-% SiO₂—pH was measured to be 9.5.

9) pH was adjusted with H₂SO₄ to 8.0 and the slurry was mixed for 30 minutes.

After the four-layered coating with SiO₂ was deposited, the particles were filtered, washed and dried at 105° C. At this stage the photostability, BET and oil adsorption measurements were performed. Subsequently, the surfaces of the formed particles were coated by introducing 0.1 w-% TMP (trimethylolpropane).

### Example 5

Titanium dioxide was prepared using a sulphate process according to the method disclosed in EP0406194B1, example 1. This product was subsequently wet milled into a slurry having TiO₂ concentration of about 250 g/l. The particle size distribution of the twice wet milled base slurry was d₁₀=0.254; d₅₀=0.455; d₉₀=0.843 μm.

A 4-layered silicon dioxide coating was manufactured onto the titanium dioxide core particles.

First, the core titanium dioxide particles were directed to the first feed vessel. The temperature of the reaction vessels was maintained at 90° C. The pH of the slurry was 9.2

Subsequently, silica was introduced into the vessel in form of water glass solution (63 g/l SiO₂), and the pH of the vessel was regulated using 25 w-% H₂SO₄ and NaOH in the following way:

1) pH was adjusted with NaOH to 9.5 and the slurry was mixed for 5 minutes.

2) Adding the first 2.5 w-% SiO₂—pH was measured to be 9.2.

3) pH was adjusted with NaOH to 9.5 and the slurry was mixed for 5 minutes.

4) pH was adjusted with H₂SO₄ to 7.3 and the slurry was mixed for 10 minutes.

5) Adding the second 2.5 w-% SiO₂—pH was measured to be 9.0.

6) pH was adjusted with NaOH to 9.5 and the slurry was mixed for 5 minutes.

7) pH was adjusted with H₂SO₄ to 7.3 and the slurry was mixed for 10 minutes.

8) Adding the third 2.5 w-% SiO₂—pH was measured to be 9.0.

9) pH was adjusted with NaOH to 9.5 and the slurry was mixed for 5 minutes.

10) pH was adjusted with H₂SO₄ to 7.3 and the slurry was mixed for 10 minutes.

11) Adding the fourth 2.5 w-% SiO₂—pH was measured to be 9.5.

12) pH was adjusted with NaOH to 9.5 and the slurry was mixed for 5 minutes.

13) pH was adjusted with H₂SO₄ to 7.3 and the slurry was mixed for 30 minutes.

After the four-layered coating with SiO₂ was deposited, the particles were cooled to 60° C. filtered, washed and dried at 105° C. At this stage the photostability, BET and oil adsorption measurements were performed. Subsequently, the surfaces of the formed particles were coated by introducing 0.1 w-% TMP (trimethylol propane).

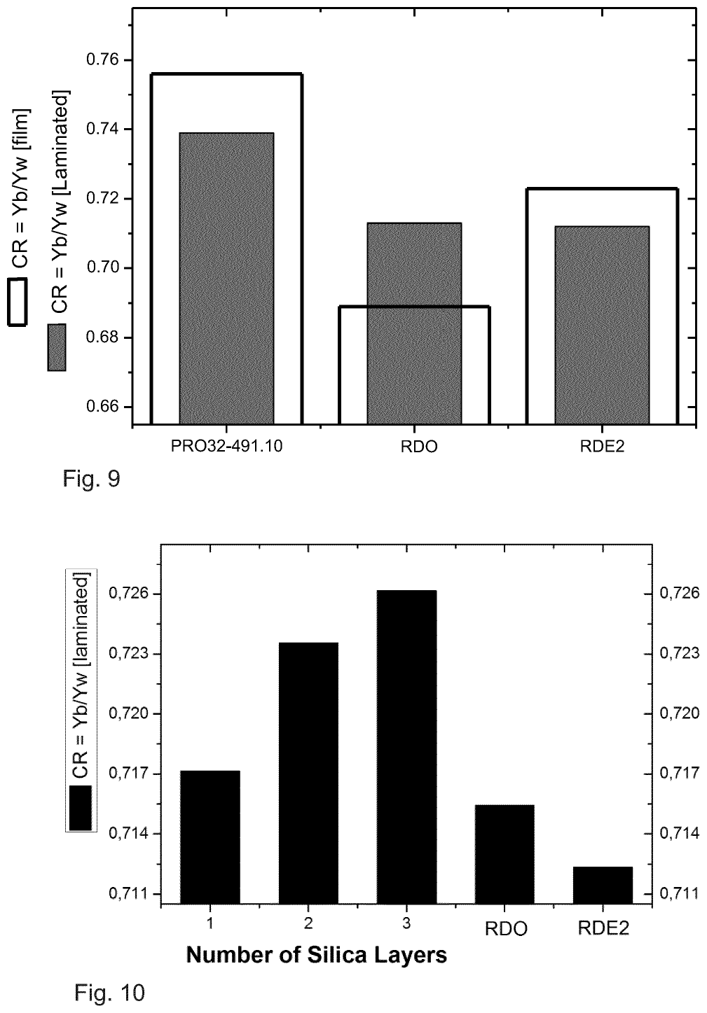

The results of the samples from this experiment (PRO32-491.10) were compared to commercially available samples RDO and RDE2 and included into a laminated film. FIG. 9 depicts the results from this comparison wherein the coated TiO₂ is included into bare (unlaminated) films and in laminated films. The contrast ratio (CR) is measured in polyurethane lamination ink with laminated films of 12 μm using Leneta 2A.

The results of the samples from examples 4 and 5 in terms of oil absorption, SiO₂ amount, undertone b*, BET and bulk density TP₀, TP₁₀₀ and TP₆₀₀ are presented in Table 2.

TABLE 2

| | Example 4 | Example 5 |
|---|---|---|
| | 65° C. | 90° C. |
| oil absorption | 24.2 | 30.7 |
| SiO₂ amount | 4.9 | 9.6 |
| b* | −6.51 | −5.98 |
| L* | 64.98 | 64.90 |
| BET | 9 | 10 |

### Example 6

The performance of multiple SiO₂ layer coated TiO₂ samples (567.3 and 567.4) were compared to single SiO₂ coated samples (RDO and RDE).

The samples included measurements made from bare films and from laminated films.

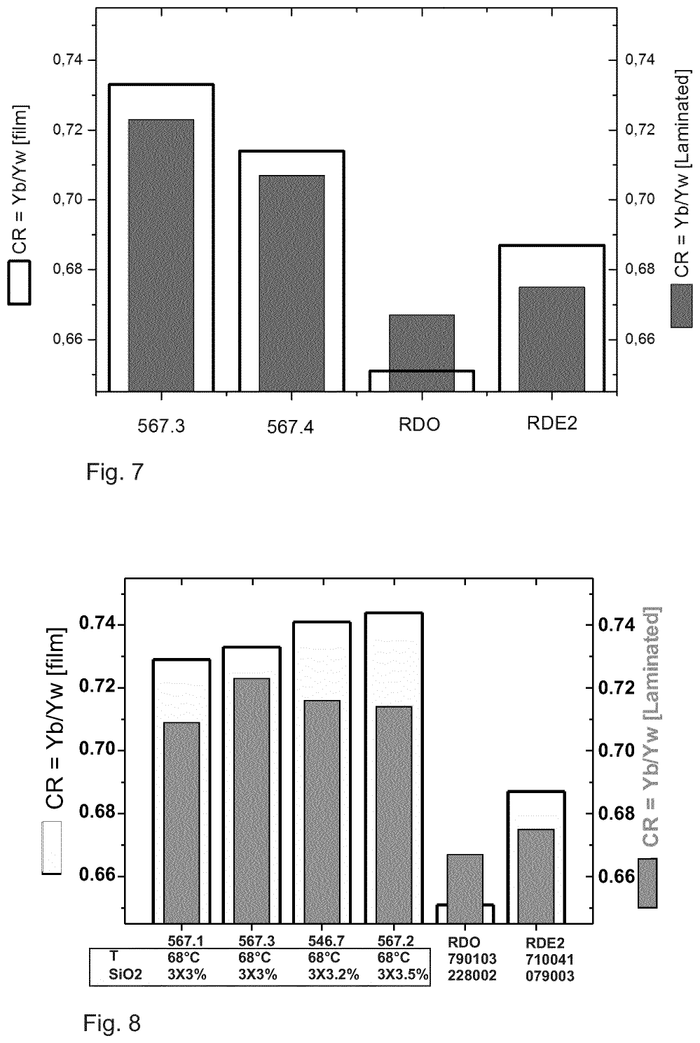

FIG. 7 shows that the multiple SiO₂ layer coated TiO₂ samples provided clearly better contrast ratio values than single layer coated samples.

### Example 7

Three layered SiO₂ coated TiO₂ samples (567.1, 567.3, 546.7 and 567.2) made with varying pH cycling were compared to single SiO₂ coated samples (RDO and RDE).

The samples included measurements made from bare films and from laminated films.

FIG. 8 shows the contrast ratio (CR) of PU lamination ink (Neorez U-471) film on OPP by gravure laminated with PE film, measured with 13-IND-068 HunterLab UltraScan XE. The multiple SiO₂ layer coated TiO₂ samples provided clearly better contrast ratio values than the single layer coated samples.

### Example 8

Three layered SiO₂ coated TiO₂ sample (3×SiO₂) was compared to single SiO₂ coated sample (RDE2). The measured results are shown in table 3.

TABLE 3

| Grade | Exp. 3xSiO₂ | TiO₂ RDE2 |
|---|---|---|
| Brightness L* - powder -Xrite i7 | 98.1 | 98.1 |
| Colour tone b* - powder - Xrite i7 | 1.6 | 2.3 |
| Tint reducing power L* - grey paste | 64.9 | 63.9 |
| Undertone b* - grey paste | −6.3 | −6.7 |
| Oil absorption (%) | 28 | 28 |
| Surface area (m²/g) | 12 | 19 |
| TiO₂ (%) | 92 | 90 |
| Inorganic surface treatment | SiO₂ | SiO₂/Al₂O₃ |
| Organic surface treatment | TMP | TMP |
| Average particle size, μm (Coulter N5) | 0.32 | 0.32 |

### Examples 9-11

Titanium dioxide was prepared using a sulphate process according to the method disclosed in EP0406194B1, example 1. This product was subsequently wet milled into a slurry having TiO₂ concentration of about 325 g/l. The particle size distribution of the wet milled base slurry was d₁₀=0.146; d₅₀=0.331 μm.

Silicon dioxide coatings of

A) 1 (1×8% SiO₂)

B) 2 (2×4% SiO₂)

C) 3 (3×2.67% SiO₂)

were manufactured onto the titanium dioxide core particles.

First, the core titanium dioxide particles were directed to the first feed vessel. The temperature of the reaction vessels was maintained at 80° C. The pH of the slurry was 9.3.

Subsequently, silica was introduced into the vessel in form of water glass solution (68 g/l SiO₂), and the pH of the vessel was regulated using 25 w-% H₂SO₄ and 30 w-% NaOH in the following ways:

For coating A:

1) Adding the 8 w-% SiO₂—pH was measured to be 9.8.

2) pH was adjusted with NaOH to 10.5 and mixed for 10 min.

3) pH was adjusted with H₂SO₄ to 7.3 and mixed for 30 min.

For coating B:

1) Adding the first 4 w-% SiO₂—pH was measured to be 9.8.

2) pH was adjusted with H₂SO₄ to 7.3 and mixed for 20 min.

3) Adding the second 4 w-% SiO₂—pH was measured to be 9.6.

4) pH was adjusted with NaOH to 10.5 and mixed for 10 min.

5) pH was adjusted with H₂SO₄ to 7.3 and mixed for 30 min.

For coating C:

1) Adding the first 2.67 w-% SiO₂—pH was measured to be 9.9.

2) pH was adjusted with H₂SO₄ to 7.3 and mixed for 20 min.

3) Adding the second 2.67 w-% SiO₂—pH was measured to be 9.6.

4) pH was adjusted with NaOH to 10.5 and mixed for 10 min.

5) pH was adjusted with H₂SO₄ to 7.3 and mixed for 20 min.

6) Adding the third 2.67 w-% SiO₂—pH was measured to be 9.3.

7) pH was adjusted with NaOH to 10.5 and mixed for 10 min.

8) pH was adjusted with H₂SO₄ to 7.3 and mixed for 30 min.

In each preparation A, B and C, the resulting slurry was cooled down to 60° C. with cold water and filtrated. The formed cake was washed and dried at 105° C.

At this stage the photostability and BET measurements were performed. Subsequently, the surfaces of the formed particles were coated by introducing 0.1 w-% TMP.

The results of the measurement are shown in Table 4 and FIG. 4.

TABLE 4

| | sample A | Sample B | Sample C |
|---|---|---|---|
| oil absorption (%) | 30.5 | 27.9 | 28.8 |
| SiO₂ amount (%) | 6.2 | 6.8 | 6.7 |
| b* | −6.23 | −6.22 | −6.25 |
| BET | 17 | 14 | 12 |
| L* | 64.84 | 64.86 | 64.21 |
| Photoactivity [ppm/h] | 4.8 | 3.3 | 2.8 |

Coating the TiO₂ pigment in SiO₂ cycles improves (decreases) the oil absorption and surface area of the particles. Also photoactivity decreases when cycles increase indicating better coverage of silica over the TiO₂ particles (FIG. 10).

### Example 12

In one example a polyurethane based lamination printing ink was prepared based on commercial NeoRez U-471.

Mill base and let down solution (21.6%)

| | |
|---|---|
| Ethanol 90%/Ethyl Acetate 10% | 236 |
| NeoRez U-471 (51%) | 174 |

Preparing of Printing Ink

Mill base

| | |
|---|---|
| Mill base solution (21.6%) | 55.6 |
| TiO₂ | 90 |

300 ml steel beaker, disc=40 mm. 3500 rpm

TiO2 61.8%, P:B=7.5:1

Let down

| | |
|---|---|
| Let down solution (21.6%) | 63.4 |
| Ethanol 90%/Ethyl Acetate 10% | 15.0 |
| total | 224 |

### TiO₂—%=40.2

P:B=3.5:1

Inks were diluted with Ethanol 90%/Ethyl Acetate 10% to viscosity of 22-24 s, measured by DIN Cup 4.

Printing inks were applied by Norbert Schläfli's Gravure ink testing machine and laminated with LL-100 benchtop laminator. The substrate was a OPP film and the lamination film was a polyethylene film. The lamination glue formula contained:

| | |
|---|---|
| Liofol UR 3966-21 | 50 g |
| Liofol LA 6074-21 | 3.8 g |
| Ethylacetate | 40 g |

1. A method for manufacturing non-flocculated discretely distributed titanium dioxide particles coated with a silica coating layer functioning as a spacer between the individual titanium dioxide particles, the method comprising the steps of:
i) forming an aqueous dispersion containing the titanium dioxide particles, wherein the mean particle size, d₅₀, of the titanium dioxide particles is in a range of 7-1000 nm,
ii) introducing to said dispersion a silicon-containing compound under constant mixing, optionally with an addition of a base, to obtain an alkaline dispersion,
iii) adding an acid to the alkaline dispersion obtained from step ii) to lower the pH to initiate precipitation of silicon oxide from the dispersion onto the titanium dioxide particles, and
iv) repeating the steps ii) and iii) at least once,
to obtain non-flocculated discretely distributed titanium dioxide particles, and preferably lowering the pH of the dispersion to a value in a range of 1.9-9.0, preferably in a range of 3-8.5, before filtering and washing the obtained product.

2. The method according to claim 1, wherein said silicon-containing compound is selected from the group consisting of water glass, silica sol, SiO₂, and an organic silicon compound, such as ortosilicate or tetraethylortosilicate, preferably water glass.

3. The method according to claim 1, wherein the base is NaOH, KOH, Na₂CO₃ or ammonia, preferably NaOH.

4. The method according to claim 1, wherein the temperature of the dispersion is in a range of 40-100° C., preferably in a range of 50-90° C., most preferably in a range of 60-85° C.

5. The method according to claim 1, wherein the pH after addition of the silicon-containing compound, and optionally the base, is in a range of 9.3-12, preferably in a range of 9.5-11.

6. The method according to claim 1, wherein the pH after addition of the acid is in a range of 4-9.3, preferably in a range of in the range of 4.3-8, more preferably in a range of 4.5-7.8, most preferably in a range of 5-7.5.

7. The method according to claim 1, wherein the acid comprises sulphuric acid, HCl, HNO₃, or an organic acid, such as formic acid, acetic acid or oxalic acid, preferably the acid is sulphuric acid.

8. The method according to claim 1, wherein the steps ii) and iii) are repeated at least two times, at least three times, at least four times, or at least five times.

9. The method according to claim 1, wherein the reaction time in the step i) and/or step ii) is at least 1 min, preferably at least 3 min.

10. The method according to claim 1, wherein the titanium dioxide concentration in a dispersion is in a range of 70-400 g/l, preferably in a range of 150-350 g/l, more preferably in a range of 200-320 g/l, such as in the range of 220-310 g/l.

11. The method according to claim 1, wherein the titanium dioxide particles are at least 80% (w/w) in rutile form, preferably at least 90% (w/w), more preferably at least 95% (w/w), most preferably at least 97% (w/w).

12. A coated titanium dioxide product, preferably manufactured by a method of claim 1, comprising at least 95% (w/w) of rutile form titanium dioxide core particles coated with a SiO₂ layer having a mean particle size of in a range of 100-1000 nm, wherein said product has ²⁹Si chemical shift peaking at (−105)-(−115) ppm in solid state NMR (nuclear magnetic resonance) spectrum indicating fully symmetric Si—O—Si bonding.

13. The coated titanium dioxide product of claim 12, wherein the titanium dioxide product has a BET surface area which is 15 m²/g or less, preferably 12 m²/g or less.

14. The coated titanium dioxide product of claim 12, wherein the titanium dioxide product has an oil absorption less than 31%, preferably less than 28%.

15. The coated titanium dioxide product of claim 12, wherein the titanium dioxide product has an undertone b* less than −6.

16. The coated titanium dioxide product of claim 12, wherein the titanium dioxide product has a tint reducing power L* (gray paste) more than 64.

17. The coated titanium dioxide product of claim 12, wherein the amount of the SiO₂ spacer coating layer is in a range of 2-4% (w/w) of the coated titanium dioxide product.

18. A coated titanium dioxide product, preferably manufactured by a method of claim 1, comprising at least 80% (w/w) of rutile form transparent titanium dioxide core particles coated with a SiO₂ layer, having a mean particle size less than 100 nm, wherein said product has ²⁹Si chemical shift peaking at (−105)-(−115) ppm in solid state NMR (nuclear magnetic resonance) spectrum indicating fully symmetric Si—O—Si bonding.

19. A printing ink composition comprising a coated titanium dioxide product of claim 12, preferably a reverse or a lamination printing ink.

20. A sunscreen composition comprising a coated titanium dioxide product of claim 12, preferably wherein the amount of the SiO₂ layer is in a range of 4-25% (w/w) of the coated titanium dioxide product.

21. A paint composition comprising a coated titanium dioxide product of 12, preferably wherein the amount of the SiO₂ layer is in a range of 2-14% (w/w) of the coated titanium dioxide product.
